(19) United States
(12) Patent Application Publication
(10) Pub. No.: US 2015/0208697 A1
(43) Pub. Date: Jul. 30, 2015

(54) APPLICATION OF LIQUIDS TO SOLID PARTICLES

(71) Applicants: Kurt Richardson, Maysville, GA (US); Julio Pimentel, Buford, GA (US); Lenka Derome, Lawrenceville, GA (US); Peter F.S. Street, Burbage (GB)

(72) Inventors: Kurt Richardson, Maysville, GA (US); Julio Pimentel, Buford, GA (US); Lenka Derome, Lawrenceville, GA (US); Peter F.S. Street, Burbage (GB)

(73) Assignee: ANITOX CORPORATION, LAWRENCEVILLE, GA (US)

(21) Appl. No.: 14/414,103

(22) PCT Filed: Jul. 14, 2012
(86) PCT No.: PCT/US2012/046821

§ 371 Date: Feb. 23, 2015

(51) Int. Cl. A23K 3/00; A23K 1/00; A23L 3/3472; A61K 47/44

(52) U.S. Cl. CPC A23K 3/00; A61K 47/44; A23K 1/004; A23L 3/3472; A23V 2002/00

(57) ABSTRACT

A method for improving the application of liquid ingredients to a solid feed ingredient or pharmaceutical agent, comprising: a) preparing a liquid composition having low cloud point containing (i) an ingredient to be added to a solid material in amounts of 1% or less, and (ii) 0.1% to 10 wt. % of a non-ionic surfactant selected from castor oil-based surfactants having 20-60 ethylene units, b) applying the liquid composition onto a solid material which is a feed ingredient or a pharmaceutical agent, by spraying the liquid composition onto the surface thereof with an average droplet size of 20-200 microns, and mixing the solid material while applying the liquid composition, whereby the coefficient of variance of the ingredient (i) is 5% or less.

### BACKGROUND OF THE INVENTION

1. Field of the Invention

Selected castor oil-based surfactants that improve mixing uniformity of preservatives and other active components used in small quantities in feed ingredients, livestock feed, aquaculture feed, companion animal food, human food, biologicals and pharmaceuticals.

2. Background

Livestock feeds and pet foods contain nutrients and preservatives that have been distributed uniformly throughout the product with the aid of a feed mixer. Factors which affect proper mixing include mixer design, mixing time, mixing speed and mixing cycle. Since the ingredients and preservatives might be provided in solid or liquid form, a mixing cycle requires a dry mix time and a wet mix time. Liquids are added after the dry mix, because liquids can reduce the dispersibility of the dry ingredients within each other, by coating the dry particles.

Every mixer and feed formulation may need a different mixing time to obtain a uniform mixture. As mixing order and feed formulations change, the mixing time requirements change also. The major advantage of a well-mixed feed is that it enhances animal performance by providing nutrients in the right proportions on a consistent basis.

The standard method for testing the uniformity of mixing or mixer performance is ASAE S303.1 (Test Procedures for Solids-Mixing equipment for Animal Feed). This method was developed by Pfost et. al. (Pfost, N. B., C. Deyoe, C. Stevens and E. Morgan.“Testing Feed Mixtures, Mixers, and Related Equipment”, Feedstuff 1976, 38: 32-46) and relies on the analytical measurement of a known marker placed in the feed. In this procedure, ten individual samples are collected from ten different sampling locations in the mixer and each sample is analyzed for a marker. Mixer uniformity is expressed as the percent Coefficient of Variance (% C.V., also called the coefficient of variation), which is the standard deviation within samples divided by the mean of those samples expressed as a percentage. Expressing coefficient of variation as a percentage allows for comparison of mixing uniformity of markers added or present in the feed. The markers used for evaluating mixing uniformity can be salt, non-protein nitrogen, calcium, DL-methionine and L-lysine, ionophores or specific metal markers used for this purpose (Microtracers Inc, San Francisco, Calif.; P. M. Clark, K. C. Behnke, and D. R. Poole, “Effects of Marker Selection and Mix Time on the Coefficient of Variation (Mix Uniformity) of Broiler Feed” J. Appl. Poult. Res. 2007, 16:464-470). Herrman and Behnke (Testing Mixing Performace, Kansas State University Extension Service Bulletin, 1994) reported the following guidelines:

TABLE 1
Guidelines for evaluating variation in mixing time

| CV | Rating | Corrective action |
|---|---|---|
| <10% | Excellent | None |
| 10-15% | Good | Increase mixing time by 25-30% |
| 15-20% | Fair | Increase mixing time by 50%, look for worn |
| | | equipment, overfilling or sequence of |
| | | ingredient addition |
| 20%+ | Poor | Possible combination of all of the above. Consult |
| | | extension personnel or feed equipment manufacturer |

The above guidelines (Table 1) suggest some possible causes for non-uniform distribution of ingredients (Wicker and Poole, “Quality Assurance as a Tool to Reduce Losses in Animal Feed Production”, Adv. Feed. Technol. 1991; 6:6-23). The Degussa Corporation, using one of its amino acids as a tracer for testing mixing uniformity found that only about half of the mixed feed samples were uniformly mixed (C.V. <10%); 30% had a C.V. of 10-20% and the remaining 20% had a C.V. of >30%. (Mixing: A Detailed Look at the Factors that Influence Mix Uniformity. Jared R. Froetschner, M.Sc. Marketing Manager, DSM Nutritional Products, Inc. Parsippany, N.J., http://nmfeed.com/Files/Posts/Portal1/4(31).pdf).

Stark conducted a study using salt and reported similar data to that of Degussa with about 42% of the mixed feed samples having a C.V. of <10%, 46% between 10% and 20%, and 12% having a C.V. of >20% (Stark, C. R., et al., 1991. On-farm feed uniformity survey. Swine Day Report No. 641. Kansas State University).

Liquid nutrients and preservatives are each added to feed at low concentrations ranging from 0.1 to 1%, so uniform dispersion of the liquid is critical in a dry feed. These liquids can include water, molasses, fat, liquid amino acid sources, organic acids, biologicals, pharmaceuticals and preservatives.

A unique property of non-ionic surfactants is the display of cloud point. This property refers to the temperature at which the surfactant phase separates and precipitates out of solution. Knowing the cloud point helps to determine the storage stability since storing formulations at temperatures significantly lower than the cloud point may result in phase separation and instability. Basically, the dissolved liquids or solids are no longer completely soluble, precipitating as a second phase giving the fluid a cloudy appearance. Generally, non-ionic surfactants show optimal effectiveness when used near or below their cloud point (Singh, Dhananjay, 2011 “Effect of Different Additives on Cloud Point of Non Ionic Surfactant,” Bachelor of Technology thesis. Department of Chemical Engineering, National Institute of Technology, Rourkela, INDIA)

When a liquid additive is cloudy or phase-separated it becomes difficult to obtain a uniform distribution of the liquid on the solid matrix. The inventors have found that selecting the correct surfactant increases stability of the liquid, since it does not show phase separation which would require mixing of the liquid prior to application, and that allows for better distribution on solid particles thus lowering the % C.V.

U.S. Pat. No. 5,591,467 demonstrated that the efficacy of a preservative in feed is directly correlated with its uniformity of application. The uniformity of application was measured by determining the % coefficient of variance. This patent discloses using 0.5% of a non-ionic surfactant to solubilize a terpene in the formulation of the preservative, with no other function of the surfactant described. The resulting % CV was less than 7%.

Surfactants are chemicals that reduce surface tension or the interfacial tension between two liquids. Surfactants play an important role in emulsifying, dispersing, spreading, cleaning, wetting, foaming and as anti-foaming agents in many practical applications. They are classified according to chemical structure and polar group: anionic, cationic, amphoretic and non-ionic. Surface activity arises because a surfactant's' structure contains two groups of contrasting solubility or polarity. In aqueous systems polar groups are known as hydrophilic, and non-polar groups as hydrophobic or lipophilic.

One property of surfactants is to act as a wetting agent, which reduces the surface tension of a liquid to promote wetting. A wetting agent allows a liquid to spread and penetrate more easily across any solid surface. When the wetting agent is applied, it causes the liquid to create particles called micelles which allow for penetration of the solid by the liquid. Wetting agents increase colorant compatibility, freeze-thaw stability and adhesion of a coating to a substrate.

Wetting is an important criterion in selecting surfactants for cleaning, coating, emulsion polymerization, pesticide applications and many others. Products which benefit from improved wetting include textile scours, laundry aids, hard-surface cleaners, dishwashing detergents, rinse aids and metal cleaners. Three distinct stages in the wetting of a solid surface can be defined. The first stage, adhesional wetting, refers to the establishment of a three-phase contact at the solid surface. Spreading wetting involves displacement of one liquid by another liquid at the solid surface. The third stage, immersional wetting represents the complete transfer of a solid particle from one fluid phase to the other (Characterization of the Wetting and Dewetting Behavior of Powders S. Chander and R. Hogg, D. W. Fuerstenau, KONA #25 (2007)).

The present invention uses non-ionic surfactants as wetting agents to help with the uniform distribution of preservatives in livestock feed and companion animal food, and it can also be applied to the pharmaceutical industry. It improves the uniformity of application of a liquid onto a dry material and reduces the variability seen upon recovery of the preservative from said material. Other ingredients added at low concentrations (0.1 to 1.0%) can also be applied using the present invention.

Non-ionic surfactants do not ionize in aqueous solution because their hydrophilic groups do not disassociate. These groups are typically alcohols, phenols, ethers, esters, or amides. A large proportion of these nonionic surfactants are made hydrophilic by the presence of a polyethylene glycol chain obtained by the polymerization of ethylene oxide. Examples of non-ionic surfactants include polyoxyethylenesorbitan monooleate (polysorbate 60), polyoxyethylenesorbitan trioleate (polysorbate 80), polyoxyethylenesorbitan monostearate, alkyltrimethylammonium bromides, dodecyltrimethylammonium bromide, hexadecyltrimethylammonium bromide, mixed alkyltrimethylammonium bromide, tetradecyltrimethylammonium bromide, benzalkonium chloride, benzethonium chloride, benzyldimethyldodecylammonium bromide, benzyldimethylhexadecylammonium bromide, benzyltrimethylammonium chloride, benzyltrimethylammonium methoxide, cetylpyridinium bromide, cetylpyridinium chloride, cetyltributylphosphonium bromide, cetyltrimethylammonium bromide, decamethonium bromide, dimethyldioctadecylammonium bromide, methylbenzethonium chloride, methyl mixed trialkyl ammonium chloride, methyltrioctylammonium chloride, n,n′,mb′-polyethylene(10)-n-tallow-1,3-diaminopropane and 4-picoline dodecyl sulfate, polyoxyethylene ethers of octyl and nonylphenols, polyethylene glycol dioleates; polyoxyalkylene laurates; polyoxyethylene ether of fatty alcohols, polyoxyethylene sorbitan monolaurate, monoesters of propyleneglycol and of the food fatty acids, stearyl-2-lactylic acid, acetic, lactic, citric, tartaric and monoacetyltartaric esters of the mono and diglycerides of food fatty acids, glycerin polyethyleneglycol ricinoleate, polyethyleneglycol esters of soybean oil fatty acids, sorbitan monostearate sorbitan tristearate, sorbitan monolaurate, sorbitan monooleate, sorbitan monopalmitate and propyleneglycol alginate.

The present invention uses ethoxylated castor oil produced by the reaction of castor oil with ethylene oxide. Ethoxylated castor oil may have various chain lengths, depending on the quantity of ethylene oxide used during synthesis. The molar ratio can vary from 1 molecule of castor oil to 1 molecule of ethylene oxide, up to 1 molecule of castor oil to 2000 molecules of ethylene oxide. These types of ethoxylated castor oil are differentiated by the nomenclature PEG-x (polyethylene glycol) castor oil, where “x” is the number of ethylene oxide molecules. (Fruijtier-Polloth, Caludia, “Safety assessment on polyethylene glycols (PEGs) and their derivatives as used in cosmetic products.” Toxicology 2005, 214:1-38; Meyer Th., J. Bohler and A. W. Frahm, “Determination of Cremophor® EL in plasma after sample preparation with solid phase extraction and plasma protein Precipitation,” Journal of Pharmaceutical and Biomedical Analysis 2001, 24:495-506). Ethoxylated castor oil emulsifiers have been widely used to solubilize water insoluble drugs for human and animal treatments. They are nonvolatile, stable compounds, which do not hydrolyze or deteriorate on storage.

Castor oil surfactants also have commercial application in pharmaceutical and food products. Cremophor 40, a commercially available product, is an ethoxylated castor oil emulsifier with 40 ethylene molecules. It is used as an emulsifying or solubilizing agent in a wide variety of cosmetic products, typically at concentrations up to 5%. It is also used in a range of pharmaceutical forms and in food-contact applications (Cosmetic ingredient review,” Final report on the safety of assessment of PEG-30, -33, -35, -36, and −40 castor oil and PEG-30 and PEG-40 hydrogenated castor oil”. Int. J. Toxicol. 1997, 16: 269-306). Cremophor EL is an ethoxylated castor oil surfactant approved for human use as a vehicle for oral and intravenous administration of water insoluble compounds. Cremophor EL is also used as a solubilizer and emulsifying agent in the foodstuff industries.

The prior art does not report selection of surfactants based on their ability to improve the uniformity of distribution of a small amount of an ingredient into a mixture. In the present invention the selection of a castor oil based surfactant improves the uniformity of distribution of a liquid onto a dry material, thus decreasing the % CV and reducing the variability of recovery of the preservative from that material.

Published patent applications refer to the use of ethoxylated castor oil surfactants in feed ingredients and complete feed. WO 99/60865 relates to the use of a surfactant-water emulsion that is added to animal feed before or after heat treatment. The emulsion helps maintain or reduce water loss during the heat treatment process. This emulsion consists of 1 to 8 parts water and 0.005 to 0.5 parts surfactant. The surfactant used has a melting point of greater than 15° C.

WO 97/28896 teaches an aqueous mixture of molasses, fat, oil, acids and water which contains an ethoxylated castor oil as a solubilizer that prevents separation of the mixture.

WO 96/11585 discloses an animal feed containing a polyethylene glycol compound that includes an ethoxylated castor oil with sixty ethoxylated molecules, which improves the nutrient value of animal feed.

WO 95/28091 describes adding ethoxylated castor oil to conventional dry animal feed, which is said to improve absorption of nutritious substances, to increase animal growth and to decrease mortality. The ethoxylated castor oil described has from 8-35 ethoxylated molecules.

U.S. Pat. No. 6,482,463 discloses an ethoxylated castor oil for animal feed to improve the availability of nutritious substances. The ethoxylated castor oil is said to aid in the formation of fat micelles in the intestinal tract, thus improving fat digestion/absorption.

The patents above describe adding ethoxylated castor oil surfactant, preferably as an emulsion, to improve the digestibility of hydrophobic substances in feeds within the animal, but as formulated they do not improve the distribution of small amounts of an active ingredient through the feed.

WO 03/096807 suggests that alkyl toluene and alkyl benzene-based surfactants increase wettability and penetrating capabilities of agricultural herbicides.

WO 02/38684 suggests the use of 1-15% wetting agent to coat a particle containing an active ingredient. The wetting agent is introduced onto the powder material as a fine mist spray.

WO 97/42836 suggests the use of wetting agents to form a coherent feed particle by spraying a fat/active ingredient emulsion that will encapsulate a feed pellet, resulting in increased pellet durability (PDI). The amount of emulsifying agent is typically in the range of 0.1 to 5 wt % of the liquid formulation.

WO/2006/024620 uses a castor oil based surfactant from 2-25 PEG units to disperse oil soluble pigments so that they can be easily applied to feed. The dispersion comprises an edible oil or fat, one or more oil-soluble pigments, and one or more non-ionic surfactants. The invention also relates to such a composition suitable for coating pellets to be used as animal feed.

EP2283733 teaches a non-ionic surfactant and an antioxidant added to ruminant feed either alone or in combination with digestion-enhancing agents to improve feedstock utilization efficiency in ruminant livestock. The disclosure relates to the encapsulation of a particulate or liquid ruminant feed additive by the non-ionic surfactant. The method increases the shelf life of the particulate feed additive.

U.S. Pat. No. 6,221,381 discloses that when non-ionic surfactants are admixed in ruminant feedstuffs at a concentration of about 0.01 to 1% (w/w) and fed to ruminants, significantly higher milk yield, increased rate of weight gain, higher efficiency in converting feed into body tissues or milk, and/or reduction in manure production follows due to improved absorption of nutrients.

EP 0363733 discloses an active substance absorbed in a microporous particle, then coated with a combination of surfactant and 5-20% of a fatty acid salt (calcium or magnesium salt). This surface active agent in combination with oil is applied by spraying on a solid mixture containing a polymer embedded with an active ingredient during mixing. The product granules obtained in this manner are encapsulated with a thin layer of water-soluble or water-dispersable non-toxic polymer which forms a film at a temperature less than 60° C.

U.S. Pat. No. 5,260,260 has a non-ionic herbicidal and surfactant blend and includes a method for improving the performance of the herbicide. A surfactant is added to liquid or dry flowable herbicides in order to help the herbicides enter the leaf surface of the weed. Once the herbicide enters a leaf surface, the herbicide can be translocated to an action site within the weed and can kill the weed. The surfactant acts as a penetrant, spreader, sticker, stabilizer, wetting agent, dispersant and defoamer. The surfactant is a nonoxynol-based surfactant.

U.S. Pat. No. 4,772,481 uses a preservative mixture of formaldehyde, formic acid, methanol, water and surfactant. The surfactant aids in the penetration of the preservative into dry feed. In addition to functioning to enhance penetration, the surfactants can also function as emulsifiers for inclusion into the premix or preservative compositions of ingredients normally insoluble therein.

U.S. Pat. Nos. 5,518,750, 5,139,779, 5,240,727 and 5,279,838 suggest the use of the surfactant sarsaponin to increase moisture levels of grains, making them more palatable to animals.

U.S. Pat. No. 5,198,253 teaches a conditioner for treating grain prior to rolling the grain into flakes so as to cause increased water uptake and gelatinization. The grain conditioner composition is an aqueous solution of a propylene glycol nonionic surfactant and water. The increase in moisture uptake is desirable to allow the production of thinner, higher volume flakes to be produced during the rolling process.

U.S. Pat. No. 3,615,653 discloses an acidic aqueous solution containing lignosulfonate and a wetting agent to aid the penetration of the solution into grain to improved starch gelatinization and nutrient utilization.

U.S. Pat. No. 3,682,653 teaches an aqueous mixture of liquid lecithin, and a food grade acid, such as propionic acid, which is capable of reducing the pH of the lecithin and thus permitting the lecithin to become water dispersable. The patent suggests that propionic acid penetrates the waxy coating of the grain and lecithin reduces surface tension of water so as to increase the penetration of moisture into the grain. The mixture contains 0.5 to 1.5% of a nonionic propylene glycol surfactant.

U.S. Pat. No. 7,134,957 uses surfactants and wetting agents to decrease microbial levels on the hide of an animal by dispersing an antimicrobial throughout the hide.

U.S. Pat. No. 7,645,464 uses an extract of African shea butter tree as a wetting agent or emulsifier. The extract can be used as a wetting agent in many applications e.g. spraying of pesticides and herbicides, dust control, etc. Furthermore it can be formulated with other surfactants, builders and ingredients normally used in detergents.

None of these patents disclose or suggest how to selected a surfactant that improves the uniformity of distribution of an active ingredient in the mixture, even though they do suggest how to select surfactants as wetting agents. The present invention shows that non-ionic surfactants, such as ethoxylated castor oil, improve the % coefficient of variance, establishing that small amounts of ingredients can be efficiently distributed into a mixture such as livestock feed, aquaculture feed, companion animal food, biological, pharmaceuticals and human food.

Various publications are referenced throughout this specification. The disclosure of each document is hereby incorporated by reference in its entirety.

### SUMMARY OF THE INVENTION

An object of the invention is to provide a method for improving the application of liquid ingredients to a solid feed ingredient or pharmaceutical agent, comprising:
a) preparing a liquid composition having low cloud point containing (i) an ingredient to be added to a solid material in amounts of 1% or less, and (ii) 0.1% to 10 wt. % of a non-ionic surfactant selected from castor oil-based surfactants having 20-60 ethylene units,
b) applying the liquid composition onto a solid material which is a feed ingredient or a pharmaceutical agent, by spraying the liquid composition onto the surface thereof with an average droplet size of 20-200 microns, and
   mixing the solid material while applying the liquid composition, whereby the coefficient of variance of the ingredient (i) is 5% or less.

Another object of the invention is to provide a feed or pharmaceutical composition obtained by a process, comprising:
a) preparing a liquid composition having low cloud point containing (i) an ingredient to be added to a solid material in amounts of 1% or less, and (ii) 0.1% to 10 wt. % of a non-ionic surfactant selected from castor oil-based surfactants having 20-60 ethylene units,
b) applying said liquid composition to a solid material which is a feed ingredient or a pharmaceutical agent by spraying the liquid composition onto the surface thereof with an average droplet size of 20-200 microns, and
   mixing the solid material while applying the liquid composition, whereby the coefficient of variance of said ingredient (i) is 0.1 to 5%.

Another object of the invention is to provide a method for improving the % C.V. obtained on liquid application of ingredients to a solid material which is a single feed ingredient, a mixture of ingredients used for livestock feed, companion animal food or human food, comprising: adding 0.1% to 10 wt. % of a non-ionic surfactant castor oil-based surfactants having 20-60 ethylene units, and spraying the liquid composition onto the surface thereof with an average droplet size of 20-200 microns while mixing.

Another object is to provide a method that improves the % C.V. of added ingredients on application of a liquid composition to the surface of a pharmaceutical for animal or human disease treatment or prevention, or as a nutritional supplement, by adding 0.1% to 10 wt. % of a non-ionic surfactant selected from castor oil-based surfactants having 20-60 ethylene units to the liquid composition.

Another object is to provide a method that improves the % C.V. on application of a liquid composition containing a preservative, nutritional supplement, biologic, drug or other material to feed or pharmaceuticals by adding 0.1% to 10 wt. % of a non-ionic surfactant selected from castor oil-based surfactants having 20-60 ethylene units to the liquid composition.

Another object is to provide a method for improving the % C.V. of a liquid composition added to a solid material, comprising:
a) preparing a low cloud point liquid composition containing (i) an ingredient to be added to a solid mater in amounts of 1% or less, and (ii) 0.1% to 10 wt. % of a non-ionic surfactant selected from castor oil-based surfactants having 20-60 ethylene units,
b) applying said liquid composition to feed ingredients, livestock feed, aquaculture feed, companion animal food, human food, biologics or a pharmaceutical, by spraying the liquid composition onto the surface thereof with an average droplet size of 20-200 microns while mixing, and
c) determining the coefficient of variance of ingredient (i) is 5% or less.

### DETAILED DESCRIPTION OF THE INVENTION

### Definitions

The primary surfactants of the invention are castor oil-based surfactants having 20-60 ethylene units in amounts up to 10 wt. %, preferably from 1 to 3 wt %. Additional surfactants selected from cationic, anionic and non-ionic wetting agents used to decrease the surface tension of liquid ingredients may replace part of the castor oil-based surfactants, in amounts from 1 to 50% of the amount of primary surfactant, preferably from 5 to 25%. These additional wetting agents include polysorbate 20, polysorbate 40, polysorbate 60, polyglyceryl ester, polyglyceryl monooleate, decaglyceryl monocaprylate, propylene glycol dicaprilate, triglycerol monostearate, sorbitan monostearate 20, sorbitan monostearate 40, sorbitan monostearate 60, sorbitan monostearate 80. The invention may include ionic surfactants such as alkyltrimethylammonium bromides, dodecyltrimethylammonium bromide, hexadecyltrimethylammonium bromide, mixed alkyltrimethylammonium bromide, tetradecyltrimethylammonium bromide, benzalkonium chloride, benzethonium chloride, benzyldimethyldodecylammonium bromide, benzyldimethylhexadecylammonium bromide, benzyltrimethylammonium chloride, benzyltrimethylammonium methoxide, cetylpyridinium bromide, cetylpyridinium chloride, cetyltributylphosphonium bromide, cetyltrimethylammonium bromide, decamethonium bromide, dimethyldioctadecylammonium bromide, methylbenzethonium chloride, methyl mixed trialkyl ammonium chloride, methyltrioctylammonium chloride, n,n′,mb′-polyethylene(10)-n-tallow-1,3-diaminopropane and 4-picoline dodecyl sulfate. Other surfactants which may be added include polyoxyethylene ethers of octyl and nonylphenols, polyethylene glycol dioleates; polyoxyalkylene laurates; polyoxyethylene ether of fatty alcohols, polyoxyethylene sorbitan monolaurate, monoesters of propyleneglycol and of the food fatty acids, stearyl-2-lactylic acid, acetic, lactic, citric, tartaric and monoacetyltartaric esters of the mono and diglycerides of food fatty acids, glycerin polyethyleneglycol ricinoleate, polyethyleneglycol esters of soybean oil fatty acids, sorbitan monostearate sorbitan tristearate, sorbitan monolaurate, sorbitan monooleate, sorbitan monopalmitate and propyleneglycol alginate.

The term “effective amount” of a compound is the amount capable of performing the function of the compound or property for which the effective amount is expressed, such as a non-toxic but sufficient amount to attain a specified % C.V. Thus an effective amount may be determined by one of ordinary skill in the art using routine experimentation.

“Weight percent” (wt. %) of a component is based on the total weight of the formulation or composition in which the component is included.

### Methods

The present invention improves the application of liquid products to feed ingredients, livestock feed, aquaculture feed, companion animal food, human food, biologicals and pharmaceuticals. These liquid products are applied by a spray nozzle providing an average droplet size of 20-200 microns. The liquid products of the present invention are generally applied in combination with a soluble carrier, other additives, and minor or major components of the mixture.

The amounts and types of surfactant will depend on the properties of the liquid ingredient to be applied. The surfactant should offer a good distribution (% CV)<10%, preferably less than 7% or 6% or 5% or 4% or 3% or 2% of the specified ingredient without any separation or change in properties of said ingredient.

The surfactant concentration in the liquid composition can vary from 0.1% to 10% preferably from 0.5 to 2%, more preferably 1 to 1.5%,

### Example 1

The objective of this study was to evaluate the stability of a commercial product (Composition “T”) in combination with different surfactants. Composition “T” is a formaldehyde-based preservative for animal feed which uses polysorbate-80 as the surfactant. For this study polysorbate-80 was replaced with ethoxylated castor oil surfactants with 40 (CO-40) or 60 (CO-60) polyethylene units. The compositions were stored at three temperatures and checked weekly for 6 weeks for cloudiness and fluidity. The temperatures were −20° C., 4° C., and 37° C. The formulations are shown in Table 2.

TABLE 2
Formulations (%) using polysorbate-80 or castor oil surfactants
used in Example 1.

| | Formalin | | | | | |
|---|---|---|---|---|---|---|
| | (37% HCHO | Poly- | | | | |
| | solution) | sorbate-80 | CO-40 | CO-60 | Inert | Total |
| Composition | 90 | 1.2 | | | 8.80 | 100 |
| “T” | | | | | | |
| Composition | 90 | | 1.2 | | 8.80 | 100 |
| #1 | | | | | | |
| Composition | 90 | | | 1.2 | 8.80 | 100 |
| #2 | | | | | | |

TABLE 3
7-day and 6-week visual results of formulations using polysorbate-80 or
castor oil surfactant.

Temperature

| | −20° C. | 4° C. | 37° C. |
|---|---|---|---|
| 7-day | | | |
| observations | | | |
| Composition “T” | cloudy (sediment at | clear | clear |
| | bottom) | | |
| Composition #1 | cloudy (sediment at | clear | clear |
| | bottom) | | |
| Composition #2 | cloudy (sediment at | cloudy | cloudy |
| | bottom) | | |
| 6-week | | | |
| observations | | | |
| Composition “T” | frozen (100%) | clear | clear |
| Composition #1 | slightly frozen (<30%) | clear | clear |
| Composition #2 | slightly frozen (<30%) | cloudy | cloudy |

The results showed that replacement of polysorbate 80 with castor oil surfactant CO-40 in composition “T” resulted in a clear product with less cloudiness at 4° C. and 37° C. At lower temperatures, castor oil surfactant CO-40 appeared to improve stability over the polysorbate-80 and castor oil C-60 compositions. (Table 3).

### Example 2

As in Example 1, polysorbate-80 from Composition “T” was replaced with castor oil surfactants having 30, 35, 40 and 60 polyethylene units (CO-30, CO-35, CO-40 and CO-60, respectively) to determine the effect on physical characteristics three days after storage at 4° C. Formulations used as shown in Table 4.

TABLE 4
Formulations (%) used on Example 2.

| | Formalin (37% HCHO | | | |
|---|---|---|---|---|
| Treatment | solution) | Inert | Surfactant | Total |
| Polysorbate - | 90 | 9.34 | 0.66 | 100 |
| 80 | | | | |
| CO-30 | 90 | 9.34 | 0.66 | 100 |
| CO-35 | 90 | 9.34 | 0.66 | 100 |
| CO-40 | 90 | 9.34 | 0.66 | 100 |
| CO-60 | 90 | 9.34 | 0.66 | 100 |

TABLE 5
Effect of refrigeration on the above formulations

| | Observation | | After 24 h at | After 3 days at |
|---|---|---|---|---|
| Treatment | at mixing* | After 1 h* | 4° C. | 4° C. |
| Polysorbate-80 | 0 | 0 | 2 | 2 |
| CO-30 | 0 | 0 | 1 | 1 |
| CO-35 | 3 | 0 | 1 | 1 |
| CO-40 | 0 | 1 | 1 | 1 |
| CO-60 | 3 | 3 | 3 | 4 |

*Scale from 0 to 4: 0 = clear, 1 = clear with separation, 2 = slightly cloudy with/without precipitates, 3 = slightly cloudy with/without separation, and 4 = cloudy with/without precipitates and/or separation.

The results in Table 5 show that the replacement of polysorbate-80 with castor oil surfactant CO-30, CO-35 or CO-40 improves the stability of composition “T” at 4° C. compared to polysorbate-80.

### Example 3

Polysorbate-80 was replaced by ethoxylated castor oil surfactant (SCO and CCO) or benzalkonium chloride. SCO-40 (CCO-40) and SCO-60 (CCO-60) are non-ionic ethoxylated castor oil surfactants from two commercial sources. Benzalkonium chloride (Benz. cl.) is a cationic surfactant. Two concentrations of surfactant were used, 0.6% and 1.0%. Formulations were stored for seven days at −17° C. and then observed for stability. Formulations used and results are shown on Table 6.

TABLE 6
Formulations (wt. %) and visual characteristics after storage at −17° C. for seven days

| | | SCO- | SCO- | CCO- | CCO- | | |
|---|---|---|---|---|---|---|---|
| | Poly- | 40 | 40 | 60 | 60 | Benz. cl. | Benz. cl |
| | sorbate-80 | 0.60% | 1.00% | 0.60% | 1.00% | 0.60% | 1.00% |
| Formalin (37% HCHO | 90.0 | 90.0 | 90.0 | 90.0 | 90.0 | 90.0 | 90. [symbol] |
| solution) | | | | | | | |
| Propionic Acid | 9.0 | 9.0 | 8.6 | 9.0 | 8.6 | 9.0 | 8.6 [symbol] |
| Surfactant | 0.60 | 0.60 | 0.60 | 0.60 | 0.60 | 0.60 | 0.6 [symbol] |
| D-limonene | 0.40 | 0.40 | 0.40 | 0.40 | 0.40 | 0.40 | 0.4 [symbol] |
| Total (g) | 100 | 100 | 100 | 100 | 100 | 100 | 10 [symbol] |
| Initial Observation* | 0 | 0 | 0 | 3 | 3 | 3 | 3 |
| After 7 days incubation at | 0 | 0 | 0 | 4 | 4 | 4 | 4 |
| −17° C.* | | | | | | | |

*Scale from 0 to 4, 0 = clear, 1 = clear with separation, 2 = slightly cloudy with/without precipitates, 3 = slightly cloudy with/without separation and 4 = cloudy with/without precipitates and/or separation. [symbol]
indicates data missing or illegible when filed

The replacement of polysorbate with SCO-40 at 0.6% or 1.0% in composition “T” improved the stability of the product at low temperatures as compared to CCO-60 and benzalkonium chloride.

### Example 4

Polysorbate-80 in composition “T” was replaced with hydrogenated castor oil (HCO-16, or ethoxylated castor oil (CO-30 or CO-40). All castor oil products were from the same manufacturer. Surfactants were used at concentrations of 0.6% and 1.0%. Formulations were stored for seven days at −17° C. and then observed for physical characteristics. The formulations and results are shown in Table 7.

TABLE 7
Formulations (%) and visual characteristics after storage at −17° C. for seven days

| | Poly- | | | | | | |
|---|---|---|---|---|---|---|---|
| | sorbate- | HCO-16 | HCO-16 | CO-30 | CO-30 | CO-40 | CO-40 |
| | 80 | 0.6% | 1.0% | 0.6% | 1.0% | 0.6% | 1.0% |
| Formalin (37% | 90.0 | 90.0 | 90.0 | 90.0 | 90.0 | 90.0 | 90.0 |
| HCHO solution) | | | | | | | |
| Propionic acid | 9.0 | 9.0 | 8.6 | 9.0 | 8.6 | 9.0 | 8.6 |
| Surfactant | 0.6 | 0.6 | 1.0 | 0.6 | 1.0 | 0.6 | 1.0 |
| D-limonene | 0.4 | 0.4 | 0.4 | 0.4 | 0.4 | 0.4 | 0.4 |
| Total (%) | 100 | 100 | 100 | 100 | 100 | 100 | 100 |
| Initial | 2 | 4 | 4 | 4 | 2 | 4 | 2 |
| Observation | | | | | | | |
| After 7 days | 2 | 4 | 4 | 4 | 2 | 4 | 2 |
| incubation at −17° C. | | | | | | | |

*Scale from 0 to 4, 0 = clear, 1 = clear with separation, 2 = slightly cloudy with/without precipitates, 3 = slightly cloudy with/without separation and 4 = cloudy with/without precipitates and/or separation.

The replacement of polysorbate with CO-40 at 0.6% or 1.0% in composition “T” improved the stability of the product at low temperature compared to HCO-16 and CO-30 (Table 7)

### Example 5

The objective of this study was to evaluate different types and concentrations of surfactants on the stability of Composition “T”. The formulations were stored for a week at the following temperatures: −17° C., 4° C., 21° C., and 54° C. followed by visually checking for physical characteristics changes. Surfactants used and formulations are shown in Tables 8 and 9, respectively.

TABLE 8
Surfactants used in Example 5

| | Surfactants | Synonyms/Chemical Names |
|---|---|---|
| 1 | T-Maz | Polysorbate-80 |
| 2 | Benzalkonium chloride | Benzalkonium chloride |
| 3 | Cavamax -W6 | Cylcohexaamylose |
| | (α-cyclodextrin) | |
| 4 | CCO-60 | Ethoxylated castor oil (CO-60) |
| 5 | CCO-35 | Ethoxylated castor oil (CO-35) |
| 6 | Lauramide-G | Na-lauroyl-L-arginine ethyl ester HCl) |
| | | glycerine |
| 7 | Lauramide-N | (N-lauroyl-L-arginine ethyl ester) |
| | | propylenglycol |
| 8 | Nonoxynol-9 (NP-9) | Nonylphenol ethoxylate |
| 9 | Octoxynol-9 (OP-9) | Octylphenoxypolyethoxyethanol |
| 10 | Propylene glycol | 1,2-Propanediol |
| 11 | PCO-30 | Ethoxylated castor oil (CO-30) |
| 12 | PCO-40 | Ethoxylated castor oil (CO-40) |
| 13 | PCO-60 | Ethoxylated castor oil (CO-60) |
| 14 | PHCO-60 | Polyethylene glycol (60) hydrogenated |
| | | castor oil |
| 15 | PHCO-16 | Hydrogenated castor oil POE-16 |
| 16 | P2000 DPS (PEG-stearate) | Polyethylene glycol (40) monostearate |
| | | flake |
| 17 | SCO-40 | Ethoxylated castor oil (CO-40) |
| 18 | Synperonic F108 | Polyethylene glycol, propoxylated |
| 19 | GS32 | Polyglyceryl-3-Disteatrate |
| 20 | A25 | Fatty alcohol ethoxylate |

TABLE 9
Formulas (%) used to prepared different concentration of surfactants in
Composition “T”

| | 0.6% | 1.0% | 2.0% |
|---|---|---|---|
| | surfactant | surfactant | surfactant |
| Formalin (37% HCHO | 90.00 | 90.00 | 90.00 |
| solution) | | | |
| Propionic acid | 9.00 | 8.60 | 7.60 |
| Surfactant | 0.60 | 1.00 | 2.00 |
| D-limonene | 0.40 | 0.40 | 0.40 |
| Total (%) | 100 | 100 | 100 |

TABLE 10
Physical characteristics of Composition “T” when using different
surfactants

| | | Temperature | | | |
|---|---|---|---|---|---|
| Surfactant | % | −17° C. | 4° C. | 21° C. | 54° C. |
| Tmaz | 0.6% | 4 | 4 | 0 | 0 |
| | 1.0% | 4 | 0 | 0 | 0 |
| | 2.0% | 0 | 0 | 0 | 0 |
| PCO-30 | 0.6% | 4 | 4 | 4 | 4 |
| | 1.0% | 2 | 2 | 2 | 0 |
| | 2.0% | 0 | 0 | 0 | 0 |
| PCO-40 | 0.6% | 4 | 4 | 0 | 0 |
| | 1.0% | 2 | 2 | 0 | 0 |
| | 2.0% | 0 | 0 | 0 | 0 |
| PCO-60 | 0.6% | 4 | 4 | 4 | 0 |
| | 1.0% | 4 | 4 | 4 | 0 |
| | 2.0% | 4 | 4 | 4 | 0 |
| PHCO-60 | 0.6% | 4 | 4 | 4 | 0 |
| | 1.0% | 0 | 0 | 0 | 0 |
| | 2.0% | 0 | 0 | 0 | 0 |
| PHCO-16 | 0.6% | 3 | 3 | 3 | 3 |
| | 1.0% | 3 | 3 | 3 | 3 |
| | 2.0% | 4 | 4 | 4 | 4 |
| P2000 | 0.6% | 4 | 4 | 4 | 0 |
| | 1.0% | 4 | 4 | 4 | 0 |
| | 2.0% | 4 | 4 | 4 | 0 |
| SCO-40 | 0.6% | 0 | 0 | 0 | 0 |
| | 1.0% | 0 | 0 | 0 | 0 |
| | 2.0% | 0 | 0 | 0 | 0 |
| Synperonic | 0.6% | 1 | 1 | 1 | 0 |
| F108 | 1.0% | 1 | 1 | 1 | 0 |
| | 2.0% | 1 | 1 | 1 | 0 |
| Benzalkonium | 0.6% | 4 | 4 | 3 | 0 |
| chloride | 1.0% | 4 | 4 | 3 | 0 |
| | 2.0% | 4 | 4 | 3 | 0 |
| Cavamax W6 | 0.6% | 4 | 0 | 4 | 0 |
| (α-cycl) | 1.0% | 3 | 4 | 0 | 0 |
| | 2.0% | 0 | 0 | 0 | 0 |
| CCO-60 | 0.6% | 4 | 0 | 0 | 0 |
| | 1.0% | 0 | 0 | 0 | 0 |
| | 2.0% | 4 | 0 | 0 | 0 |
| CCO-35 | 0.6% | 0 | 0 | 0 | 0 |
| | 1.0% | 2 | 0 | 0 | 0 |
| | 2.0% | 1 | 0 | 0 | 0 |
| Lauramide-G | 0.6% | 4 | 1 | 2 | 0 |
| | 1.0% | 4 | 1 | 2 | 0 |
| | 2.0% | 4 | 1 | 2 | 0 |
| Lauramide- N | 0.6% | 0 | 0 | 0 | 0 |
| | 1.0% | 0 | 0 | 0 | 0 |
| | 2.0% | 0 | 2 | 2 | 0 |
| NP-9 | 0.6% | 4 | 2 | 0 | 0 |
| | 1.0% | 4 | 2 | 0 | 0 |
| | 2.0% | 4 | 4 | 4 | 0 |
| OP-9 | 0.6% | 4 | 4 | 0 | 0 |
| | 1.0% | 4 | 1 | 2 | 0 |
| | 2.0% | 4 | 4 | 1 | 0 |
| Propylene | 0.6% | 4 | 4 | 1 | 0 |
| Glycol | 1.0% | 4 | 4 | 1 | 0 |
| | 2.0% | 4 | 4 | 1 | 0 |
| GS32 | 0.6% | 4 | 4 | 4 | 4 |
| | 1.0% | 4 | 4 | 4 | 4 |
| | 2.0% | 4 | 4 | 4 | 4 |
| A25 | 0.6% | 4 | 4 | 4 | 4 |
| | 1.0% | 4 | 4 | 4 | 4 |
| | 2.0% | 4 | 4 | 4 | 0 |

* Scale from 0 to 4, 0 = clear, 1 = clear with separation, 2 = slightly cloudy with/without precipitates, 3 = slightly cloudy with/without separation and 4 = cloudy with/without precipitates and/or separation.

Twelve of the twenty surfactants tested demonstrated similar characteristics as polysorbate-80 when incorporated in “Composition T” and stored at different temperatures (Table 10). Ethoxylated castor oil surfactants with 30 to 60 ethylene units had similar characteristics as polysorbate-80. Ethoxylated castor oil with 16 polyethylene units was not as effective as surfactants with 30 to 60 polyethylene units.

### Example 6

In this study, “Composition T” was prepared using one of seven surfactants listed on Table 10 at a 0.6 wt. % concentration. Formulations (2 kg/MT) were applied to 1000 g of poultry feed in a lab scale feed mixer equipped with a liquid spray application system. This system provided mixing uniformity similar to normal field conditions as described by Wicker and Poole in Table #1.

After treatment, ten 2-gram samples were taken from the mixer for determining the concentration of the active ingredient and calculating the uniformity of distribution of “Composition T” (% C.V). The concentration of the active ingredient in the feed samples was determined by chemical analysis.

TABLE 11
Surfactants used for feed application studies in Example 6.

| Surfactants | Synonyms/Chemical Names |
|---|---|
| T-Maz | Polysorbate-80 |
| CCO-60 | Ethoxylated castor oil (CO-60) |
| CCO-35 | Ethoxylated castor oil (CO-35) |
| PCO-30 | Ethoxylated castor oil (CO-30) |
| PCO-40 | Ethoxylated castor oil (CO-40) |
| PCO-60 | Ethoxylated castor oil (CO-60) |
| SCO-40 | Ethoxylated castor oil (CO-40) |

TABLE 12
Percent Coefficient of Variance (% CV) of the recovery of
formaldehyde from composition “T” formulated with different
ethoxylated castor oil surfactants described in Table 11.

| | | | Polysorbate- |
|---|---|---|---|
| Set #1 | CCO-60 | CCO-35 | 80 |
| avg ± std | 2.18 ± 0.19 | 2.14 ± 0.18 | 2.10 ± 0.24 |
| % CV | 8.64 | 8.19 | 11.61 |
| | | | Polysorbate- |
| Set #2 | PCO-30 | PCO-40 | 80 |
| avg ± std | 1.75 ± 0.09 | 1.87 ± 0.20 | 1.80 ± 0.21 |
| % CV | 5.34 | 10.53 | 11.84 |
| | | | Polysorbate- |
| Set #3 | PCO-60 | SCO-40 | 80 |
| avg ± std | 1.56 ± 0.18 | 1.66 ± 0.18 | 1.77 ± 0.29 |
| % CV | 11.60 | 11.00 | 16.08 |

Results showed that the use of ethoxylated castor oil surfactants with 30 to 60 ethylene units gave a better distribution of the preservative composition (formaldehyde) than polysorbate-80.

It will be apparent to those skilled in the art that a number of modifications and variations may be made in the present invention without departing from the scope of the invention. It is intended that the specification and examples be considered as exemplary only, with a true scope and spirit of the invention being indicated by the following claims.

1. A method for improving the % C.V. of a liquid composition added to a solid material, comprising:
a) preparing a low cloud point liquid composition containing (i) an ingredient to be added to a solid material in amounts of 1% or less, and (ii) 0.1% to 10 wt. % of a non-ionic surfactant selected from castor oil-based surfactants having 20-60 ethylene units, and
b) applying said composition to feed ingredients, livestock feed, aquaculture feed, companion animal food, human food, biologics or a pharmaceutical, by spraying the liquid composition onto the surface thereof with an average droplet size of 20-200 microns while mixing, and
determining that the coefficient of variance of ingredient (i) is 5% or less.

2. The method of claim 1, wherein (b) comprises: a single feed ingredient, a mixture of ingredients used for livestock feed, aquaculture feed, companion animal food or human food.

3. The method of claim 1, wherein (b) contains a pharmaceutical used for animal or human diseases prevention/treatment, or as a nutritional supplement.

4. The method of claim 1, wherein composition (a) contains a preservative, nutrition supplement, pharmaceutical, or biological applied in liquid form to a feed or pharmaceutical product.

5. The method of claim 1, wherein the non-ionic surfactant is an ethoxylated castor oil surfactant with 25 to 60 ethylene units.

6. The method of claim 1, wherein the non-ionic surfactant is from 0.5 to 2% by weight of composition (a).

7. The method of claim 1, wherein composition (a) is applied by spray atomization.

8. The method of claim 1, wherein composition (a) is applied by a hydraulic spray system.

9. The method of claim 1, wherein the C.V. is 4% or less.

10. A method for improving the application of liquid ingredients to a solid feed ingredient or pharmaceutical agent, comprising:
c) preparing a liquid composition having low cloud point containing (i) an ingredient to be added to a solid material in amounts of 1% or less, and (ii) 0.1% to 10 wt. % of a non-ionic surfactant selected from castor oil-based surfactants having 20-60 ethylene units,
d) applying the liquid composition onto a solid material which is a feed ingredient or a pharmaceutical agent, by spraying the liquid composition onto the surface thereof with an average droplet size of 20-200 microns, and
mixing the solid material while applying the liquid composition, whereby the coefficient of variance of the ingredient (i) is 5% or less.

11. The method of claim 10, wherein the non-ionic surfactant is from 0.5 to 2% by weight of composition (a).

12. The method of claim 10, wherein the coefficient of variance of the active ingredient is 4% or less.

13. A feed or pharmaceutical composition obtained by a process, comprising:
c) preparing a liquid composition having low cloud point containing (i) an ingredient to be added to a solid material in amounts of 1% or less, and (ii) 0.1% to 10 wt. % of a non-ionic surfactant selected from castor oil-based surfactants having 20-60 ethylene units,
d) applying said liquid composition to a solid material which is a feed ingredient or a pharmaceutical agent by spraying the liquid composition onto the surface thereof with an average droplet size of 20-200 microns, and
mixing the solid material while applying the liquid composition, whereby the coefficient of variance of said ingredient (i) is 0.1 to 5%.

14. The composition of claim 13, wherein the surfactant is from 0.5 to 2% by weight of composition (a).

15. The composition of claim 13, wherein the coefficient of variance of the active ingredient is 4% or less.
